(19) United States
(12) Patent Application Publication
(10) Pub. No.: US 2011/0270073 A1
(43) Pub. Date: Nov. 3, 2011

(54) ELECTRON SPIN RESONANCE IMAGING SCANNER

(75) Inventors: Jan-Henrik Ardenkjaer-Larsen, Frederiksberg C (DK); Jonathan Alan Murray, Dousman, WI (US); Fraser John Laing Robb, Aurora, OH (US); Ralph Eugene Hurd, Menlo Park, CA (US); Victor Taracila, Orange Village, OH (US)

(73) Assignee: GENERAL ELECTRIC COMPANY, SCHENECTADY, NY (US)

(21) Appl. No.: 12/769,967

(22) Filed: Apr. 29, 2010

(51) Int. Cl. A61B 5/055

(52) U.S. Cl. 600/410

(57) ABSTRACT

An electron paramagnetic resonance imaging (EPRI) system includes a resistive magnet driven by a power supply such as a power supply module to generate radio frequency signals in a substantially coherent polyphase perfect sequence scheme. The EPRI system further includes image acquisition and processing electronics configured to generate, acquire, quantify and map pO2 information associated with a free radical agent in vivo and having a resonance line width that is sensitive to oxygen and in response to the radio frequency signals without imparting harmful heating effects to a corresponding human or animal body.

### BACKGROUND

The invention relates generally to electron paramagnetic resonance imaging (EPRI), and more particularly to an electron spin resonance imaging system that accommodates clinical applications such as, without limitation, pO2 mapping.

EPRI is an imaging modality based on the imaging of exogenous electron paramagnetic resonance probes. It is thus a combination of a scanner and a contrast agent. Molecules suitable for use with EPRI to map pO2 have been developed and are well known. Mapping pO2 is of particular importance regarding, without limitation, radiotherapy planning in cancer treatment and wound healing/amputation of extremities. EPRI however has limitations. These limitations include sensitivity, fast relaxation time of the spin (large band width required), and high frequency of detection. These factors mean that the spatial resolution is limited, and that absorption of radio frequency energy limits the sensitivity and ability to quantify pO2.

Pulsed EPRI to date has been limited to small animal and has progressed from mice to rats as electronics has become faster. Solutions for scaling up further have employed continuous wave acquisition with the penalty of lower sensitivity. Low-power pulsed schemes of the past have also been suffering from low sensitivity and also from loss of signal-to-noise. Further, no solutions are presently known in EPRI to overcome known problems regarding radio frequency penetration and transmit/receive switch dead time.

It would therefore be advantageous to provide an EPRI system that overcomes the foregoing fundamental limitations of EPRI, among others. The EPRI system should overcome EPRI limitations caused by heating of the patient by the radio frequency field, EPRI limitations caused by the inhomogeneity of the radio frequency field, and EPRI limitations caused by long dead time of the transmit/receive switch.

### BRIEF DESCRIPTION

Briefly, in accordance with one embodiment, an electron paramagnetic resonance imaging (EPRI) system comprises

a resistive magnet driven by a power supply module to generate a static magnetic field in the range 0-20 mT. This field will dictate the resonance frequency of the electron spins in the range 0-560 MHz. The resistive magnet can be of several designs, e.g. a solenoid, Helmholtz or saddle coil. The magnet is further equipped with three orthogonal gradient coils allowing spatial encoding of the spins by applying field gradients.

The EPRI system further comprises a radio frequency signal source and pulse programmer configured together with the resistive magnet and gradient coils to generate a substantially polyphase perfect sequence scheme and excite a free radical agent in vivo there from without imparting harmful heating effects to a human or animal body;

a transmit/receive switch designed to isolate the radio frequency pulses from a corresponding detection system characterized in allowing interleaved radio frequency pulses and data acquisition according to the substantially polyphase perfect sequence scheme;

image acquisition and processing electronics configured to acquire EPR signals coherent with the pulse sequence; and

software to quantify and map pO2 information associated with the free radical agent in vivo and having a resonance line width that is sensitive to oxygen.

According to another embodiment, an electron spin resonance imaging system is configured to generate a polyphase perfect sequence scheme allowing an essentially homogeneous radio frequency field to penetrate a human body such that pO2 information associated with a free radical agent in vivo and having a resonance line width that is sensitive to oxygen is generated, acquired, quantified and mapped via corresponding signal acquisition and processing electronics in response thereto without imparting harmful heating effects to a corresponding human or animal body.

### DRAWINGS

These and other features, aspects, and advantages of the present invention will become better understood when the following detailed description is read with reference to the accompanying drawings in which like characters represent like parts throughout the drawings, wherein:

FIG. 1 is a simplified system diagram illustrating an electron paramagnetic resonance imaging (EPRI) system that operates to quantify pO2 information associated with a human body according to one embodiment of the invention; and

FIG. 2 is a more detailed system diagram illustrating an electron paramagnetic resonance imaging system that operates to quantify pO2 information associated with a human body according to one embodiment of the invention.

While the above-identified drawing figures set forth alternative embodiments, other embodiments of the present invention are also contemplated, as noted in the discussion. In all cases, this disclosure presents illustrated embodiments of the present invention by way of representation and not limitation. Numerous other modifications and embodiments can be devised by those skilled in the art which fall within the scope and spirit of the principles of this invention.

### DETAILED DESCRIPTION

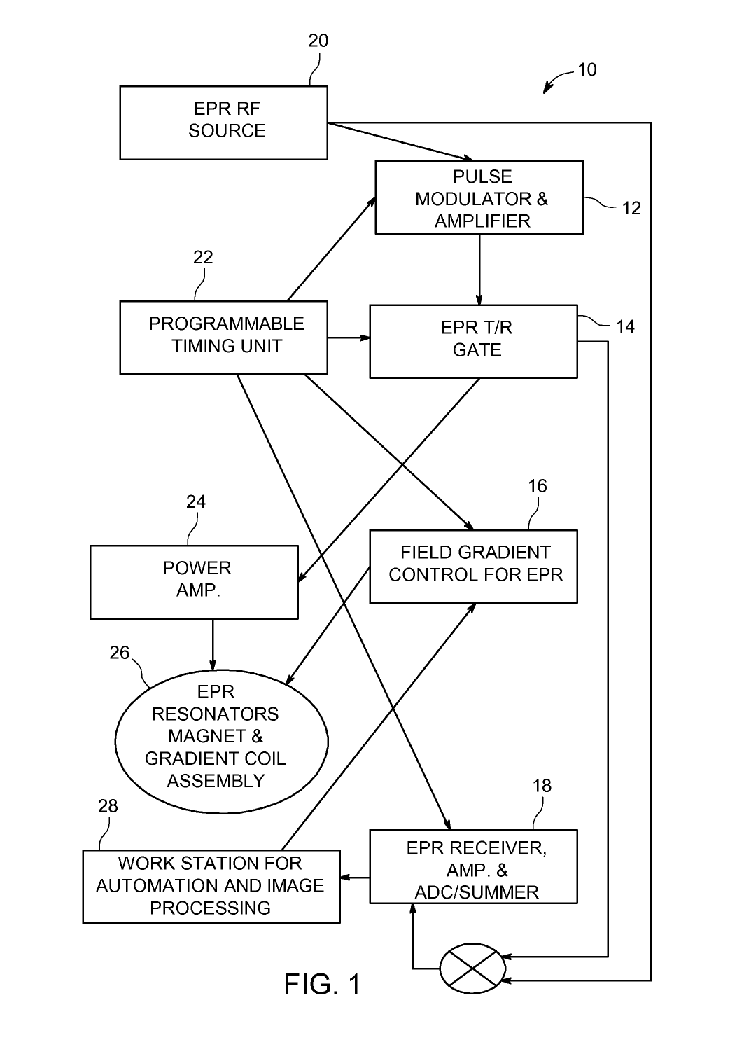

FIG. 1 is a simplified high order system diagram illustrating an electron paramagnetic resonance imaging (EPRI) system 10 that operates to quantify pO2 information associated with a human body according to one embodiment of the invention. EPRI system 10 comprises an EPR pulse modulator and amplifier module 12 having an output coupled to the input of an EPR transmit/receive gate 14, an EPR field gradient controller 16, and an EPR receiver, amplifier and ADC/summer 18. EPRI system 10 further comprises a radio frequency source 20, a programmable timing unit 22, a power amplifier 24, EPR resonators, magnet and gradient coil assembly 26, and a work station for automation and image processing 28. The magnet and gradient coil assembly 26 comprises a primary magnet for generating a static magnetic field and gradient coils for generating gradient magnetic fields.

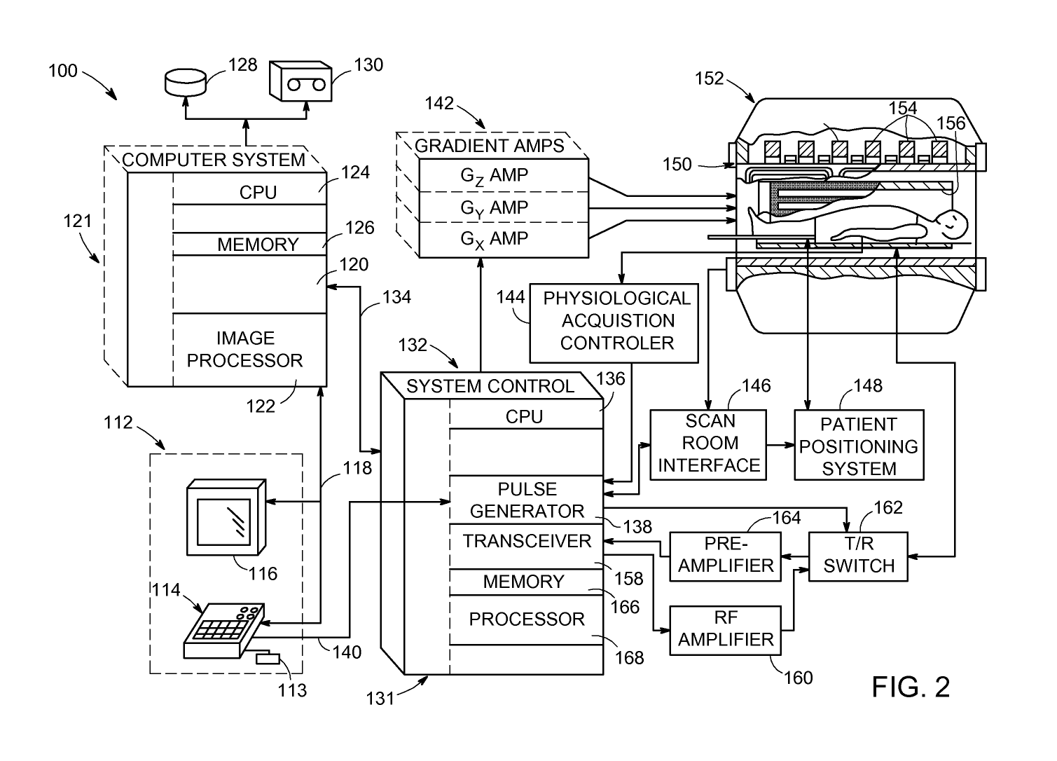

FIG. 2 is a more detailed system diagram illustrating an electron paramagnetic resonance imaging system 100 that operates to quantify pO2 information associated with a human body according to one embodiment of the invention. EPRI system 100 is controlled from an operator console 112, which includes a keyboard or other input device 113, a control panel 114, and a display screen 116. The console 112 communicates through a link 118 with a separate computer system 120 that enables an operator to control the production and display of images on the display screen 116. The computer system 120 includes a number of modules which communicate with one another through a backplane 121. These include an image processor module 122, a CPU module 124 and a memory module 126, known in the art as a frame buffer for storing image data arrays. The computer system 120 is linked to disk storage 128 and tape drive 130 for storage of image data and programs, and communicates with a separate system control 132 through a high speed serial link 134. The input device 113 can include a mouse, joystick, keyboard, track ball, touch activated screen, light wand, voice control, or any similar or equivalent input device, and may be used for interactive geometry prescription.

The system control 132 includes a set of modules connected together by a backplane 131. These include a CPU module 136 and a pulse generator module 138 which connects to the operator console 112 through a serial link 140. It is through link 140 that the system control 132 receives commands from the operator to indicate the scan sequence that is to be performed. The pulse generator module 138 operates the system components to carry out the desired scan sequence and produces data which indicates the timing, strength and shape of the RF pulses produced, and the timing and length of the data acquisition window. The pulse generator module 138 connects to a set of gradient amplifiers 142, to indicate the timing and shape of the gradient pulses that are produced during the scan. The pulse generator module 138 can also receive patient data from a physiological acquisition controller 144 that receives signals from a number of different sensors connected to the patient such as ECG signals from electrodes attached to the patient. And finally, the pulse generator module 138 connects to a scan room interface circuit 146 which receives signals from various sensors associated with the condition of the patient and the magnet system. It is also through the scan room interface circuit 146 that a patient positioning system 148 receives commands to move the patient to the desired position for the scan.

The gradient waveforms produced by the pulse generator module 138 are applied to the gradient amplifier system 142 comprising Gx, Gy and Gz amplifiers. Each gradient amplifier excites a corresponding physical gradient coil in a gradient coil assembly 150 to produce the magnetic field gradients used for spatially encoding acquired signals. The gradient coil assembly 50 forms part of a magnet assembly 152 comprising a polarizing magnet 154 and a whole-body RF coil 156. A transceiver module 158 in the system control 132 produces pulses which are amplified by an RF amplifier 160 and coupled to the RF coil 156 by a transmit/receive switch 162. The resulting signals emitted by the excited nuclei in the patient may be sensed by the same RF coil 156 and coupled through the transmit/receive switch 162 to a preamplifier 164. The amplified EPR signals are demodulated, filtered and digitized in the receiver section of the transceiver 158. The transmit/receive switch 162 is controlled by a signal from the pulse generator module 138 to electrically connect the RF amplifier 160 to the coil 156 during the transmit mode and to connect the preamplifier 164 to the coil 156 during the receive mode. The transmit/receive switch 162 can also enable a separate RF coil (for example, a surface coil) to be used in either transmit or receive mode.

An Electron Paramagnetic Imaging system imposes new hardware challenges when compared to its MRI counterpart. One of the major difficulties is the fast switching from the transmit phase to receive phase. While in MRI timing is measured in 10-100 microseconds, in EPRI this would have to be in tens of nanoseconds. The switching in MRI is done by using PIN diodes, which turn on (off); and time constant is of the order of microseconds.

Receive coils in ESR need to be able to receive while current in the transmit coil is not completely attenuated (due to eddy current). If the same coil is used for receive and transmit, the eddy current effect needs to be subtracted from the total signal so only the useful sample ESR signal is processed. According to one embodiment, the transmit coil needs to be very well decoupled from the receive coil so that there is no inductive coupling between transmitter and receiver. The only coupling is of resistive nature and occurs through the patient.

The simplest coil configuration would contain two elements: transmit loop and receive loop, transmit loop and receive saddle (or vice versa) etc. Particular embodiments may employ various types of coil shapes which decouple well from each other to accommodate specific applications. One embodiment employs an array of transmit coils and a coil array of receive coils—all being inductively decoupled from each other. One example would be a central circular loop transmitter and three equally shaped loops equidistant from each other used for receiving. Each receiver loop would be inductively decoupled from the transmitter loop and as well as the other two receiver loops (utilizing triple double-spiral interleaved transformer). The area of transmitter sensitivity partially (or totally) must superpose with the area of receiver sensitivity. A three element receive array will increase the SNR and accelerate the signal acquisition.

The EPR signals picked up by the RF coil 156 are digitized by the transceiver module 158 and transferred to a memory module 166 in the system control 132. A scan is complete when an array of raw k-space data is rearranged into separate k-space data arrays for each image and each component coil to be reconstructed, and each of these is input to a central processing unit 168 which operates to Fourier transform the data into an array of image data according to one embodiment. This image data is conveyed through the communication link 134 that may be for example, and Ethernet link, to the computer system 120 where it is stored in memory, such as disk storage 128. In response to commands received from the operator console 112, this image data may be archived in long term storage, such as one the tape or disk drive 130, or it may be further processed by the image processor 122 and conveyed to the operator console 112 and presented to the display 116.

The EPRI system 100 may further be equipped with a receive coil array that picks up the EPR signals. Such coil arrays are well-known in the art and include whole body arrays as well as partial body arrays, such as head coil arrays, cardiac coil arrays, and spine coil arrays. According to one aspect, parallel imaging may be employed wherein a region or volume of interest is sampled with an array of RF receive coils. In this regard, the embodiments described herein are not limited to a particular coil array type or orientation.

In further explanation, an EPRI scanner comprises a resistive magnet driven via a standard gradient amplifier module. This allows the imaging field to be settable anywhere from 0 T (Tesla) to about 20 mT which defines the highest achievable resonance frequency. For example, 10.7 mT using EPRI corresponds to about 300 MHz, which equates to about 7 T for 1 H (proton) using MRI, while 21.4 mT using EPRI corresponds to about 600 MHz.

When an EPRI scanner is not operational, the magnetic field is off, eliminating any need for active shielding of the stray field(s). The magnet can be a simple high-order compensated solenoid, or it can be a more open Helmholtz type coil. This makes a large bore easily accommodated, e.g. >70 cm, and provides flexibility in terms of radio frequency coils. A patient can be, for example, lying perpendicular to the main field or could even be standing. One advantage of this approach is that the main field does not have to be prescribed. The signal, e.g. radio frequency, chain is sufficiently broad band that any frequency, e.g. in the range of about 200 MHz to about 400 MHz can be chosen. This allows the exact frequency to be chosen depending on the particular application, e.g. whole body, brain, liver, extremity . . . ). It may also provide a degree of freedom during signal acquisition by field switching.

Since EPRI is limited by the specific-absorption-rate (SAR), the signal-to-noise-ratio (SNR) to a first approximation is independent of magnetic field. The magnetization increases linearly with the magnetic field. The SAR however increases with the square of the magnetic field (frequency) and the square of the radio frequency magnetic field. Thus, if the magnetic field is doubled to double the magnetization, the radio frequency excitation must be reduced to half to ensure that SAR is not exceeded and thus the SNR remains unchanged. The SNR also depends linearly on the detection frequency, e.g. induction factor, but so does the noise voltage when the sample noise is dominating.

Gradients for spatial encoding associated with EPRI are static during signal acquisition, and therefore do not require a specification for slew rate. The gradients therefore do not require shielding as eddy currents are not an issue when using EPRI. This improves gradient strength and high performance, e.g. large gradients, can be achieved with standard gradient drivers. The required gradient strength is similar to current MRI requirements, and no more than 10 mT/m.

A problem of low field imaging associated with EPRI is the concomitant field associated with large gradients relative to the main static field. The concomitant field causes geometric distortions, which need to be corrected in post-processing. This limitation speaks in favor of the highest possible magnetic field strength, reduced field-of-view and low spatial resolution.

Detection schemes according to particular embodiments of the invention described as follows herein are unique to EPRI. Due to the short relaxation time of the electron spin, there is no possibility of gradient switching during the free-induction-decay. The gradients are thus static and projections are acquired in 3D (three dimensions). The electron spin magnetization needs to be almost fully excited continuously during the spatial encoding and signal averaging in order to maximize the sensitivity. This feature has not been possible using any known detection schemes, and either very low flip angles or long repetition times have had to be employed.

An EPRI signal acquisition scheme according to one embodiment employs a radio frequency source and pulse programmer to generate radio frequency signals in a substantially coherent pulse sequence scheme such that pO2 information associated with a free radical agent in vivo with a human body and having a resonance line width that is sensitive to oxygen is acquired, quantified and mapped there from. Such radicals are known in the prior art. According to another embodiment, an EPRI signal acquisition scheme employs a radio frequency source and pulse programmer to generate substantially coherent transmission of a traveling wave or parallel transmit radio frequency pulses such that pO2 information associated with a free radical agent in vivo with a human body and having a resonance line width that is sensitive to oxygen is acquired, quantified and mapped there from. One substantially or fully coherent polyphase perfect sequence scheme that may be employed most preferably includes Frank pulses. Other substantially or fully coherent polyphase perfect sequence schemes (e.g. phase modulated pulse sequences) with similar effect to the Frank pulses that may be employed according to the principles described herein include without limitation, Chu pulses, among others. Frank pulse and Chu pulse schemes have been demonstrated for example in NMR applications. Frank pulses and Chu pulses are known and described in the art; and so further details regarding these pulse schemes are not presented herein to preserve brevity and enhance clarity in describing the embodiments discussed herein. The use of a Frank pulse scheme allows semi-continuous excitation and acquisition with very low transmit energy to minimize SAR, and effectively achieve a large saturation degree (e.g. 5-20%, or even higher). A Frank pulse scheme employed with EPRI is believed to possibly also allow T₁ₑ contrast to be used by acquiring one image at high saturation and one image at low saturation.

EPRI using fully or substantially fully coherent signal acquisition schemes such as described herein advantageously reduces the required transmit power by many orders of magnitude (e.g. >3). A pulse of e.g. 5 ns would generally be required to yield the desired bandwidth of 50-100 MHz. A large flip angle, e.g. 60°, would require a large radio frequency magnetic field amplitude. A low duty cycle (long repetition time) would be necessary to stay within SAR limitations, and SNR would be lost. The use of Frank pulses however achieves the same bandwidth by phase modulation of the pulses that each are of very low amplitude (e.g. pulse angle of less than one degree for the individual pulses). The desired EPRI signal acquisition is then interleaved with the Frank pulses.

EPRI is known to have a long dead time when using a high transmit power. This shortcoming is however overcome to a large extent by the power reduction achieved when using a Frank pulse acquisition scheme. A waveguide antenna in combination with orthogonal local antennas was found to improve the isolation between transmit and receive switching to further overcome the foregoing long dead time. Otherwise, the EPRI signal transmission and receive chains may be identical to high field MRI chains, or otherwise achieved using state-of-the-art radio frequency electronics. In high field MRI (7 T), parallel transmit has demonstrated the ability of providing a much improved radio frequency magnetic field homogeneity. Due the low power of the coherent pulse sequence scheme, the transmit-receive switch can be optimized for dead time as it does not need to accommodate the usual large transmit power of kW involved other pulse sequence schemes.

In summary explanation, EPRI embodiments described herein employ recent developments in NMR/MRI technology to overcome several of the fundamental limitations of EPRI. EPRI presently is limited by the heating of the patient by the radio frequency field, and this limitation is largely overcome by use of special pulse sequence schemes, e.g. Frank pulses, during EPRI according to one embodiment. Further, EPRI presently is limited by the inhomogeneity of the radio frequency field, and this limitation is largely overcome by use of traveling wave excitation or parallel transmit schemes during EPRI according to one embodiment. EPRI presently is also limited by a long dead time associated with the transmit/receive switch, and this limitation is overcome by use of special pulse sequence schemes alone or in combination with traveling wave excitation or parallel transmit schemes during EPRI according to one embodiment. Traveling wave excitation and parallel transmit schemes are known and described in the art; and so further details regarding such schemes are not described herein in order to preserve brevity and enhance clarity in understanding the principles discussed herein with respect to particular embodiments of the invention.

While only certain features of the invention have been illustrated and described herein, many modifications and changes will occur to those skilled in the art. It is, therefore, to be understood that the appended claims are intended to cover all such modifications and changes as fall within the true spirit of the invention.

1. An electron paramagnetic resonance imaging (EPRI) system comprising:
a resistive magnet driven by a power supply to generate a static magnetic field;
orthogonal gradient coils;
a radio frequency signal source and pulse programmer configured together with the resistive magnet and orthogonal gradient coils to generate a substantially coherent polyphase perfect sequence scheme and excite a free radical agent in vivo there from without imparting harmful heating effects to a human or animal body; and
image acquisition and processing electronics configured to generate, acquire, quantify and map pO2 information associated with the free radical agent in vivo and having a resonance line width that is sensitive to oxygen.

2. The EPRI system according to claim 1, wherein the substantially coherent polyphase perfect sequence scheme comprises Frank pulses.

3. The EPRI system according to claim 1, wherein the the substantially coherent polyphase perfect sequence scheme comprises Chu pulses.

4. The EPRI system according to claim 1, wherein the substantially coherent polyphase perfect sequence scheme is generated in a frequency range from about 0 Hz to about 600 MHz.

5. The EPRI system according to claim 1, further comprising an imaging field strength from about 0 Tesla (T) to about 21.4 mT.

6. The EPRI system according to claim 1, wherein the resistive magnet comprises a simple high-order compensated solenoid or a more open Helmholtz type coil.

7. The EPRI system according to claim 1, wherein the gradient coil system comprises a gradient strength similar to those present with current MRI requirements, and no more than about 10 mT/m.

8. The EPRI system according to claim 1, wherein the resistive magnet is driven via a gradient amplifier module to generate a corresponding electron spin magnetization that is substantially fully excited continuously during spatial encoding and signal averaging to maximize EPRI sensitivity.

9. The EPRI system according to claim 1, wherein the radio frequency signals comprise traveling waves or parallel transmit schemes to substantially minimize radio frequency field inhomogeneity during imaging.

10. The EPRI system according to claim 1, wherein the radio frequency signals comprise traveling waves or parallel transmit schemes in combination with Frank pulses to substantially minimize receive/transmit switch times during imaging.

11. The EPRI system according to claim 1, further configured to provide interleaved transmitting and receiving with about 100 ns intervals.

12. The EPRI system according to claim 1, further configured to provide interleaved transmitting and receiving with about 50 ns intervals.

13. The EPRI system according to claim 1, further configured to provide interleaved transmitting and receiving with about 10 ns intervals.

14. An electron spin resonance imaging system configured to generate a substantially coherent polyphase perfect sequence scheme allowing a substantially homogeneous radio frequency field to penetrate a human body such that pO2 information associated with a free radical agent in vivo and having a resonance line width that is sensitive to oxygen is generated, acquired, quantified and mapped via corresponding signal acquisition and processing electronics in response thereto without imparting harmful heating effects to a corresponding human or animal body.

15. The electron spin resonance imaging system according to claim 14, wherein the substantially coherent polyphase perfect sequence comprises Frank pulses.

16. The electron spin resonance imaging system according to claim 14, wherein the the substantially coherent polyphase perfect sequence comprises Chu pulses.

17. The electron spin resonance imaging system according to claim 14, wherein the substantially coherent polyphase perfect sequence is generated in a frequency range from about 0 Hz to about 600 MHz.

18. The electron spin resonance imaging system according to claim 14, wherein the imaging field strength is from about 0 Tesla (T) to about 21.4 mT.

19. The electron spin resonance imaging system according to claim 14, further comprising a resistive magnet selected from one of a simple high-order compensated solenoid and a more open Helmholtz type coil.

20. The electron spin resonance imaging system according to claim 19, further comprising a gradient amplifier, wherein the resistive magnet and gradient amplifier together generate a gradient strength similar to those associated with current MRI requirements, and no more than about 10 mT/m.

21. The electron spin resonance imaging system according to claim 20, wherein a corresponding resistive magnet field generates a corresponding electron spin magnetization that is substantially fully excited continuously during spatial encoding and signal averaging to maximize electron paramagnetic resonance imaging sensitivity.

22. The electron spin resonance imaging system according to claim 14, further comprising a traveling wave antenna or parallel transmit coil configured to substantially minimize radio frequency field inhomogeneity during imaging.

23. The electron spin resonance imaging system according to claim 14, further comprising a traveling wave antenna or parallel transmit coil configured to substantially minimize receive/transmit switch times during imaging.
